(19) United States
(12) Patent Application Publication
(10) Pub. No.: US 2004/0131561 A1
(43) Pub. Date: Jul. 8, 2004

(54) Strips for treating teeth

(76) Inventor: Gerald McLaughlin, Port Jefferson Station, NY (US)

Correspondence Address: KLARQUIST SPARKMAN, LLP, 121 SW SALMON STREET, SUITE 1600, PORTLAND, OR, 97204, US

(21) Appl. No.: 10/472,107

(22) PCT Filed: Mar. 14, 2002
(86) PCT No.: PCT/US02/07965

(51) Int. Cl. A61K 7/20

(52) U.S. Cl. 424/53

(57) ABSTRACT

A device is provided for the treatment of a subject's teeth. In one embodiment, the device includes a treatment agent delivery means and a support means to which the delivery means is attached for delivering and retaining the treatment agent. In one embodiment, the device includes a substratum which is formed from a curviplaner band of non-porous material.

### CROSS REFERENCE TO RELATED APPLICATION

This application claims priority from U.S. Provisional Application No. 60/275,965 filed Mar. 14, 2001, which is incorporated herein by reference.

### Field

A device for treating teeth, and a method for its use, are described.

### BACKGROUND

Teeth generally become more darkly pigmented with age and exposure to materials such as tea and coffee. It has long been a goal of dentistry to provide a means to safely and effectively reverse this darkening process. Historically there are two approaches to the problem. The first involves removing pigmentation that has adhered onto the surface of the teeth. The conventional techniques commonly use abrasives, sometimes augmented with solvents. While rapidly effective, these techniques have the disadvantage of only being able to remove external stains, leaving all internal pigmentation unchanged. Thus, the whitening effect is extremely limited.

A second technique involves using oxidizing agents to penetrate into the tooth structure and bleach out the undesired pigmentation. The active agents are usually either weak solutions of carbamide peroxide or hydrogen peroxide. Typically, the application of the whitening agent utilizes either custom or stock trays that are shaped to hold the bleaching agent against the teeth to be whitened. The trays are filled with gel, liquid peroxide, or other aqueous solution and worn for long periods of time, sometimes even overnight. After a series of lengthy treatments the teeth usually begin to show the desired whitening effect. The length of the treatment can be discouraging and expensive.

While effective on both external and internal discolorations, one major problem encountered with custom and stock dental trays and the aqueous solutions stems from the materials used. All aqueous solutions of peroxide (“wet” peroxides) begin to break down and lose effectiveness with time. One particularly useful agent utilized in such compositions is formed from the combination of water and granular urea peroxide. When granular urea peroxide is isolated from moisture and humidity it has an extremely long shelf life, but once mixed with water it breaks down to urea and hydrogen peroxide and immediately begins to degrade. Although this degradation is desirable during use since it is the reaction products that cause the bleaching effect, it is undesirable during shipping and storage. An attempt to overcome this limitation has been made by adding gelling agents such as Carbopol™, Pemulan™, and the like, to the urea peroxide solution. Although the gels extend the useful life of the peroxide, they also slow its effectiveness during use. Some products cannot be shipped during certain times of the year or over weekends as they loose too much potency during the shipping period due to temperature variations. Thus, most of the products currently on the market require use within a short time after manufacture and often require refrigeration during storage.

Another problem with moist whitening agents is that they cannot be conveniently carried for fear of leakage from the packaging. Leakage of a bleach material into the surrounding area of a pocketbook, pocket, or briefcase can inconvenience the carrier.

Also, in general, the most effective application of conventional whitening agents is by placement in a custom-formed tooth stent or tray. Unfortunately, this normally requires a visit to the dentist and requires the subject to endure the tooth impression procedure. In addition, the cost of the extra material and labor can be significant.

Alternatively, some people use the pre-mixed gels in a stock or non-custom tray. Unfortunately, the fit of stock trays to the teeth is imprecise and the bleaching gel rapidly leaks from the tray. For this reason, the effectiveness of stock trays currently in use is limited: after a very short time the bleaching agent will leak out leaving insufficient active ingredient in the tray. In addition to being inefficient, this configuration causes the subject to swallow a much larger amount of bleaching agent than necessary.

One solution to this problem is provided by U.S. Pat. No. 6,274,122, which discloses the use of a dry treatment agent in a trough that substantially surrounds the facial, occlusal, and lingual surfaces of at least one of a subject's teeth. However, a trough-type device is somewhat bulky and is uncomfortable to some users. Additionally, a trough-type device may not be useable by subjects with dental appliances on the lingual side of their teeth due to interference of the dental appliance with the tooth treatment device. Also, it is always desirable to provide a device that can be manufactured more easily and less expensively.

### SUMMARY

A device is provided for the treatment of a subject's teeth. A subject's teeth can include natural teeth or artificial dental structures, such as dentures, as well as the surrounding tissue. Use of the device can reverse the darkening process of a subject's teeth.

In one embodiment the device includes a treatment agent, treatment agent delivery means, and a support means to which the delivery means is attached for positioning and retaining the treatment agent.

In another exemplary embodiment of such a device, the device includes a substratum, which is formed from a curviplaner band of non-porous material, and a permeable layer that is attached to the substratum. A treatment layer that contains a treatment agent is disposed between the substratum and the permeable layer. The permeable layer allows an aqueous solution to pass through to the treatment layer and in turn allows the treatment agent to pass through to a subject's teeth. However, before the aqueous solution is added the permeable layer prevents a substantial portion of the treatment agent from passing through the permeable layer.

In another embodiment the device includes a substratum, which is formed from a curviplaner band of non-porous material, and a retaining layer attached to the substratum. A permeable layer is attached to the retaining layer. A treatment layer is disposed between the permeable layer and the retaining layer and includes a dry, solid bleaching agent.

In another embodiment the device includes a substratum formed from a curviplaner band of non-porous polymeric material and a permeable layer attached to the substratum. A treatment layer that contains a treatment agent is disposed between the substratum and the permeable layer. The treatment layer includes a composition of a bleaching agent and a water swellable agent in dry form that is free between the substratum and the permeable layer. The permeable layer allows an aqueous solution to pass through to the treatment layer and in turn allows the bleaching agent to pass through to a subject's teeth. However, before the aqueous solution is added the permeable layer prevents a substantial portion of the treatment agent from passing through the permeable layer.

A method of using a device for the treatment of a subject's teeth is also provided. In one embodiment the method includes providing a device including a non-porous substratum, a permeable layer, and a dry, solid powder bleaching agent disposed between the substratum and the permeable layer. An aqueous solution is added to device and the device is placed against only the lingual or buccal surfaces either before or after the addition of the aqueous solution.

### BRIEF DESCRIPTION OF THE DRAWINGS

FIG. 1 is a perspective view of a device for the treatment of teeth in accordance with one embodiment of the present invention.

FIG. 2 is a cross section taken along line 2-2 of FIG. 1.

FIG. 3 is a cross section of a second alternative embodiment similar to the one illustrated in FIG. 2, but further illustrating a retaining layer and an adhesive layer.

FIG. 4 is a cross section of the device illustrated in FIG. 1, but further illustrating the device after addition of an aqueous solution.

FIG. 5 is a horizontal cross section illustrating how the device is positioned with respect to a subject's teeth.

FIG. 6 is a cross section taken along the line 6-6 of FIG. 5.

FIG. 7 is a vertical cross section of a third embodiment of the invention similar to the one shown in FIG. 6, but further illustrating a retention member.

FIG. 8 is a horizontal cross section of a fourth embodiment of the invention having retention arms.

FIG. 9 is cross section of the device taken along line 9-9 in FIG. 8.

FIG. 10 is a horizontal cross section of a fifth embodiment of the present invention.

### DETAILED DESCRIPTION

As used herein and in the appended claims, the singular forms “a,” “and,” and “the” include plural referents unless the context clearly dictates otherwise. Thus, for example, reference to “a tooth” includes a plurality of teeth and reference to “the compound” includes reference to one or more compounds and equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices, and materials are now described.

All publications mentioned herein are incorporated herein by reference in full for the purpose of describing and disclosing the materials and methodologies that are described in the publications which might be used in connection with the presently described invention. The publications discussed throughout the text are provided solely for their disclosure prior to the filing date of the present application. Nothing herein is to be construed as an admission that the inventor is not entitled to antedate such disclosure by virtue of prior invention.

In practice, one side of the device is used to deliver a treatment agent by positioning that side against at least one of a subject's teeth. The device can be laminated, e.g., comprised of two or more layers. The side of the device and surface of each layer that faces a subject's teeth during use is referred to as the active side of the device or active surface of a layer. Also, because in use these portions will generally face in the direction of either the buccal or lingual surfaces of a subject's teeth, this side or surface may be referred to as the buccal or lingual side or surface when appropriate. The opposite side or surface is referred to as the non-active side or surface because in use these portions will generally face in a direction away from the treated surfaces of a subject's teeth.

The layers of the device can include, for example, a substratum, an adhesive layer, a retaining layer, a permeable layer, and a treatment layer. The device need not include each layer, and may include other layers. Additionally, each layer may itself be comprised of multiple layers or sublayers.

The substratum can be used as a base layer of material to support the other layers. The substratum has a curviplaner shape. A curviplaner shape includes substantially planer shapes that may be formed from an initially flat strip of material where the strip is substantially straight or forms an arcuate shape in the horizontal direction and where the strip is substantially straight in the vertical direction. A substratum with a curviplaner shape is not in the form of a dental tray or trough. Thus, the “U” shaped device disclosed in McLaughlin's U.S. Pat. No. 6,274,122 is not curviplaner.

A retaining layer can be attached to the substratum to retain the treatment layer containing the treatment agent. The treatment layer can be coated onto the substratum or retaining layer, or can be disposed freely between the substratum or the retaining layer and the permeable layer. The permeable layer can prevent a substantial amount of the treatment layer and treatment agent from escaping from the device before an aqueous solution is introduced. Once an aqueous solution is introduced, the permeable layer allows the treatment agent to pass. The permeable layer can be stretchable so that when the aqueous solution swells the treatment layer the permeable layer can accommodate the swelled treatment layer and so that it can extend closer to or make greater contact with the subject's teeth to allow more effective delivery of the treatment agent to the subject's teeth. The permeable layer can be attached to the substratum or can be attached to any other layer such that the treatment layer is disposed between the permeable layer and the layer to which it is attached. If the permeable layer is used with a retaining layer and a treatment layer disposed therebetween, the envelope created by the permeable and retaining layers can be removably attached to the substratum by an adhesive layer. In this way, the treatment agent could be stored separate from the substratum and purchased separately if the subject desired to reuse the substratum. The layers can be attached together throughout the device, or some or all of the layers can be attached in an area that includes a majority of the area of the device. Alternatively, the layers can be attached together in an area that includes a minority of the area of the device.

The device formed by these layers can be pre-formed into an arcuate shape to fit either or both rows of a subject's teeth, or to partially fit either or both rows. The device also can be initially flat, but be conformable to an arcuate shape to fit either or both rows of a subject's teeth, or to partially fit either or both rows. When the device is deformed to conform to a subject's teeth, the deformation can either be permanent or non-permanent. The device also can have horizontal edges and a width between those edges sufficient to cover teeth in either or both rows of a subject's mouth. The device also can have vertical edges and a distance between those edges sufficient to cover one or more teeth in a row of teeth.

The device does not need any special feature to position the device against a subject's teeth and retain the device in a suitable position to deliver the treatment agent. The device can simply be placed against the teeth to be treated and held in place by the subject's body, for example, the subject's lips or tongue. However, a retention member to retain the device in position against a subject's teeth can be provided. The retention member can be formed by an extension of a layer of the device or can be comprised of one or more arms extending from a surface of the device.

The treatment layer of the device contains a treatment agent to treat a subject's teeth or surrounding tissue. The treatment agent can be, for example, a whitening agent, a palliative agent, a tooth decay preventative agent, a tooth sensitivity reduction agent, a flavoring agent, an anti-microbial agent, or a combination of such agents. The treatment agent can be a dry powder. The treatment layer can also include other agent such as a water swellable agent.

The drawings and their following descriptions illustrate merely exemplary embodiments of a device and method for the treatment of teeth. They are not intended to limit the scope of the claims.

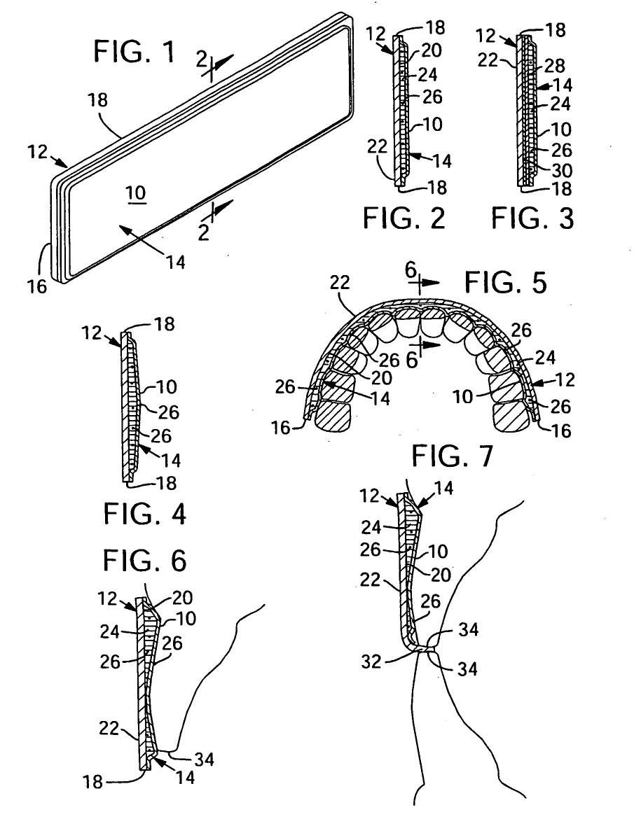

FIG. 1 illustrates a perspective view of a laminated device for the treatment of teeth. The device includes substratum 12 and permeable layer 14. The substratum 12 has horizontal edges 18 and vertical edges 16. The permeable layer 14 has an active surface 10. Also included, though not visible in this view, is treatment layer 24 including treatment agent 26.

The substratum 12 can be formed from a non-porous material, preferably a non-toxic material such as a polymeric material, for example, plastic, syrene-ethylene butylene-styrene (SEBS) polymer, polystrene foam, polyethylene foam, polyurethane foam and polyolefin foam. The substratum 12 can also be formed of another non-porous material such as foil or wax. In this view substratum 12 is flat, however, in use the substratum and any other layers of the device can be conformable to an arcuate shape to fit the upper or lower rows, or both rows, of a subject's teeth (see FIG. 5). The device also can be conformable to fit variations in the vertical surfaces of a subject's teeth. The deformation required to conform the substratum 12 can be permanent or non-permanent. Substratum 12 can be pre-formed to an arcuate shape to fit the curve of a subject's arch.

The substratum 12 has an active surface 20 and a non-active surface 22 (see FIG. 2). The substratum 12 also has two vertical edges 16 and two horizontal edges 18 (only one of each edge can be seen in FIG. 1). The width defined by horizontal edges 18 may be great enough to treat two rows of teeth, or only one. The distance between vertical edges 16 may be great enough to treat one or more teeth.

FIG. 2 illustrates a cross section of the device illustrated in FIG. 1. Treatment layer 24 and treatment agent 26 are disposed between substratum 12 and permeable layer 14. Permeable layer 14 allows the penetration of an aqueous solution such as water, but does not allow a substantial portion of treatment layer 24 in its dry state to pass through the permeable layer. As shown in FIGS. 1 and 2, permeable layer 14 is attached to active surface 20 near vertical edges 16 and along the horizontal edges 18 of substratum 12 to envelop treatment layer 24. The permeable layer 14 need only be attached adjacent one horizontal edge 18 of substratum 12. Additionally, permeable layer 14 can be attached to substratum 12 at other points, for example, along a line down the center of the permeable layer. Alternatively, permeable layer 14 can be attached to the surface of edges 18 and 16 or to non-active surface 22. These attachments can be by any means known to one of skill in the art, such as glue, cement, or a heat-seal. Preferably these substances are non-toxic.

Permeable layer 14 can be composed of a woven fabric. A “woven” fabric is a fabric that has been prepared by interlacing fibers or threads such as on a loom. Examples of woven fabrics include silk, rayon, or cotton. Permeable layer 14 can also be a non-woven fabric such as polypropylene fibers. Alternatively, permeable layer 14 can be a thin sheet of a water impermeable material that includes holes for facilitating the passage of water, such as a sheet of perforated plastic. Permeable layer 14 can also be made from an open-celled foam or a porous film.

Permeable layer 14 can be either thick or thin. A thin permeable layer may be about 13 mils-100 mils thick. A thick layer may be about 1-3 mm thick. A very thick permeable layer 14 may be thicker that 3 mm (e.g., an open celled sponge). The thickness of permeable layer 14 is based on its ability to allow treatment agent 26 to migrate to the subject's teeth and to retain the water swellable agent of treatment layer 24 once an aqueous solution is added (see below). One of skill in the art will be readily be able to determine an appropriate thickness of permeable layer 14.

Permeable layer 14 can be stretchable so that it can increase its surface area to cover the teeth of the subject and to accommodate the swelling of treatment layer 24. Permeable layer 14 can also include excess material to facilitate coverage of the teeth when treatment layer 24 is expanded by the addition of an aqueous solution. Thus, permeable layer 14 can be designed to have more surface area than treatment layer 24 or substratum 12.

Treatment layer 24 can include a composition containing one or more treatment agents 26. Treatment layer 24 can be free within the enclosure created by the attachment of substratum 12 to permeable layer 14 or, alternatively, the composition of treatment layer 24 can include adhesives, such as glue or cement, to attach the treatment layer to substratum 12. Preferably, the glue or cement is non-toxic. If treatment layer 24 is adhered to substratum 12, the grains of treatment agent 26 may be exposed to allow the treatment agent to be solubulized upon addition of an aqueous solution.

A treatment agent 26 includes any pharmaceutical, bleaching, or other dental agent, a nutrition supplement, or other biocompatible compound capable of improving the condition of the teeth or gingival such as an antioxidant, an anti-caries compound, a compound to decrease tooth sensitivity or an anti-microbial agent.

A “bleaching agent” is any agent or compound that whitens the teeth. Examples of suitable bleaching compounds include an oxygen radical generating agent such as metal ion free peroxides, organic peroxides, and metal ion containing peroxides. Specific, non-limiting examples of bleaching agents suitable for use with the invention are redox agents such as monopersulfate, Oxone, ammonium persulfate, potassium persulfate, potassium monopersulfate, potassium peroxymonosulfate, potassium bisulfate, potassium sulfate, and potassium peroxidisulfate. Additional specific, non-limiting examples of bleaching agents suitable for use with the device of the invention are the peroxide class of bleaching agents such as hydrogen peroxide, carbamide peroxide, urea peroxide, sodium percarbonate, sodium perborate, calcium hydroxide, potassium chlorate, magnesium carbonate and perhydrol urea. To whiten the teeth of a subject, the device would include a therapeutically active amount of the bleaching agent. A “therapeutically active amount of a bleaching agent” as used herein means a quantity of the bleaching agent sufficient to whiten the teeth of a subject, such as a mammal, for example, a human.

Assays to determine a therapeutically active amount of a bleaching agent are known in the art. For example, stained extracted teeth can be used to measure a whitening effect using the device provided with a treatment layer 24 including varying amounts of a bleaching agent. Other assays, such as an analysis of the effect of a bleaching agent on the soft tissues, can also be used in the determination of a therapeutically active range of concentrations of a bleaching agent.

Treatment agent 26 can be in a dry form. A “dry form” of treatment agent 26 should generally be less than 40% water by weight, preferably, less than 5% water by weight. A dry bleaching agent can be used in amounts so that about 35% to 100% by weight of the composition of the treatment layer comprises the active bleaching agent before the addition of the aqueous solution. In one embodiment, the treatment layer includes an active bleaching agent as about 40% by weight of the treatment layer before the addition of the aqueous solution. It should be noted that the percentage by weight of the bleaching agent may vary depending on the weight of any inactive ingredients included in the dry composition. The bleaching agent can be 3% to 20% by weight after the addition of the aqueous solution. If the bleaching agent is carbamide peroxide, carbamyl peroxide or perhydrol urea, the concentration of the bleaching agent can be from about 5% to about 20% or from about 10% to about 15% by weight after the addition of the aqueous solution. In one embodiment, the bleaching agent is hydrogen peroxide which is about 3%, 5%, or 10% by weight after addition of the aqueous solution.

A treatment agent 26 for decreasing tooth sensitivity includes any compound that lowers the susceptibility of a tooth to stimulation such as temperature or pressure. Compounds to decrease tooth sensitivity include, but are not limited to, potassium nitrate, citric acid, citric acid salts, sodium fluoride, and strontium chloride.

A treatment agent 26 that is an anti-microbial agent includes, for example, chlorhexadine, tetracycline, cetyl pyridinium chloride, benzalkonium chloride, cetyl pyridinium chloride, cetyl pyridinium bromide, methyl benzoate, and propyl benzoate.

A palliative treatment agent 26 for periodontal tissues includes, for example, aloe, eugenol, corticosteroid and vitamin E. Pigments, sweeteners, colors, and flavors can also be incorporated into the composition.

Treatment layer 24 can include a treatment agent 26 for administering fluoride, such as a fluorine providing salt, which can prevent cavities. Such materials are characterized by their ability to release fluoride ions in water. Agents for administering fluoride include, for example, inorganic metal salts such as sodium fluoride, potassium fluoride, and tin fluoride such as stannous fluoride or stannous chlorofluoride, sodium fluorosilicate, ammonium fluorosilicate and sodium monofluorophosphate.

Treatment layer 24 can further include a gelling agent. A “gelling agent” is an agent that forms a semisolid suspension of small inorganic or large organic molecules upon addition of an aqueous solution. The aqueous solution interpenetrates the inorganic or large organic molecules in order to form the gel. Gelling agents include, for example, alpha starch, agar, hydroxyethyl cellulose, mangrot seed, hydroxymethyl cellulose, sodium polyacrylate, sodium polyacrylamide, poly-N-vinylpyrrolidone, poly-vinyltoluenesulfonate, poly-sulfoethyl acrylate, poly-2-hydroxyethyl acrylate, poly-vinylmethyloxazolidinone, hydrolyzed polyacrylamide, polyacrylic acid, copolymers of acrylamide and acrylic acid and alkali metal salts of such of the polymers that contain sulfonate or carboxylate groups. The addition of these components to dental creams, such as toothpaste, is well known in the art.

Treatment layer 24 can further include a dry, solid, water-insoluble, physiologically unobjectionable, water-swellable agent. A “water swellable” agent is any material that increases its volume upon exposure to an aqueous solution, such as a polymeric sorbent, for example, sodium polyacrylate, sodium polyacrylamide, poly-N-vinylpyrrolidone, poly-vinyltoluenesulfonate, poly-sulfoethyl acrylate, poly-2-hydroxyethyl acrylate, poly-vinylmethyloxazolidinone, hydrolyzed polyacrylamide, polyacrylic acid, copolymers of acrylamide and acrylic acid, and alkali metal salts of such of the polymers as contain sulfonate or carboxylate groups (see U.S. Pat. No. 3,926,891; U.S. Pat. No. 3,699,103, U.S. Pat. No. 5,693,411, all herein incorporated by reference in their entirety), or a naturally occurring water-swellable agent, such as mangrot seed, ground root of the buuk plant, cotton and sponge. The solution used to swell the water-swellable agent can be water, mouthwash, juice such as aloe vera juice, a dilute salt solution, or a combination of these examples.

Treatment layer 24 can further include a catalytic agent. A “catalytic agent” is a compound or molecule that accelerates the action of the treatment agent, such as the whitening action of the bleaching compound, without being consumed in the reaction. The catalytic agent can accelerate the release of oxygen radicals from an oxygen radical generating agent, for example, activated charcoal, platinum, platinum salts, copper, copper salts, palladium, palladium salts, silver and silver salts. Of particular use with the subject invention is the commercially activated charcoal Centaur™, produced by Calgon, Inc.

Treatment layer 24 can also include an abrasive material such as dicalcium phosphate (e.g., see U.S. Pat. No. 5,171,564, herein incorporated by reference), for example, dicalcium phosphate dihydrate, anhydrous dicalcium and calcium pyrophosphate. Other abrasives include siliceous materials, for example, silica abrasives, such as precipitated amorphous hydrated silica, and alumina abrasives, such as alumina trihydrate, aluminum silicate, calcined alumina and bentonite.

In FIG. 3, permeable layer 14 is attached to retaining layer 28, which is in turn attached to active surface 20 of substratum 12 with adhesive layer 30. The adhesive layer 30 can be a glue or tacky substance. A non-hardening tacky substance such as a gum might be used so that the attached retaining layer 28 and permeable layer 14 can be removable from substratum 12.

In FIG. 4, an aqueous solution, such as water or saliva, has been introduced through permeable layer 14 to contact treatment layer 24 causing a portion of treatment layer 24 to swell and treatment agent 26 to solubilize. In turn, permeable layer 14 has stretched to accommodate the swelling. Before the addition of the aqueous solution, a buffer zone may exist between permeable layer 14 and the subject's teeth. After the addition of the aqueous solution, the swelling and stretching of treatment layer 24 and permeable layer 14 can cause the treatment layer and permeable layer to expand closer to the teeth of a subject to deliver treatment agent 26. However, if the swelling is not sufficient to close the distance between permeable layer 14 and the teeth of a subject, the device can simply be moved from its initial position to a position closer to the subject's teeth, for example, the subject can manipulate the device by hand. Additionally, once solubulized, treatment agent 26 is able to pass through permeable layer 14, allowing the treatment agent to treat the teeth of a subject.

In FIG. 5, the layers of the device conform to an arcuate horizontal cross section to fit the subject's arch. The device is shown after the addition of an aqueous solution with active surface 20 of substratum 12 and active surface 10 of permeable layer 14 facing the buccal surfaces of the subject's teeth. The layers of the device can be pre-formed to such an arcuate shape, or the device can be initially flat as in FIG. 1, but be conformable to such an arcuate shape. A pre-formed device can also be conformable to fit a wider range of arcuate shapes. If the device is conformable, the deformation required to conform the device can be permanent or non-permanent.

In FIG. 6, the vertical cross section of the substratum 12 has is substantially strainght. Non-active surface 22 of the substratum 12 faces away from the subject's tooth while active surface 20 faces towards the buccal surfaces of the subject's tooth. Permeable layer 14 is attached to substratum 12 and treatment layer 24 is disposed between the two layers. Treatment agent 26 has partially migrated through permeable layer 14 to contact the subject's teeth. The device illustrated can be held in place by a part of the subject's body, such as a lip or finger.

The alternative embodiment shown in FIG. 7 includes a retention member 32. The majority of the vertical cross section of substratum is straight, but near the occlusal surfaces 34 of the subject's teeth the extension of the substratum forming retention member 32 curves in the lingual direction. If the device were used to treat the lingual surface of a subject's teeth, retention member 32 would extend in the buccal direction. Methods to form substratum 12 to have such a shape are well known. Retention member 32 extends such that its major surfaces generally face the occusal surfaces 34 of a subject's teeth. Thus, by biting down, a subject can cause occlusal surfaces 34 of his or her teeth to contact retention member 32 and thereby retain the device in his or her mouth and in position to deliver treatment agent 26.

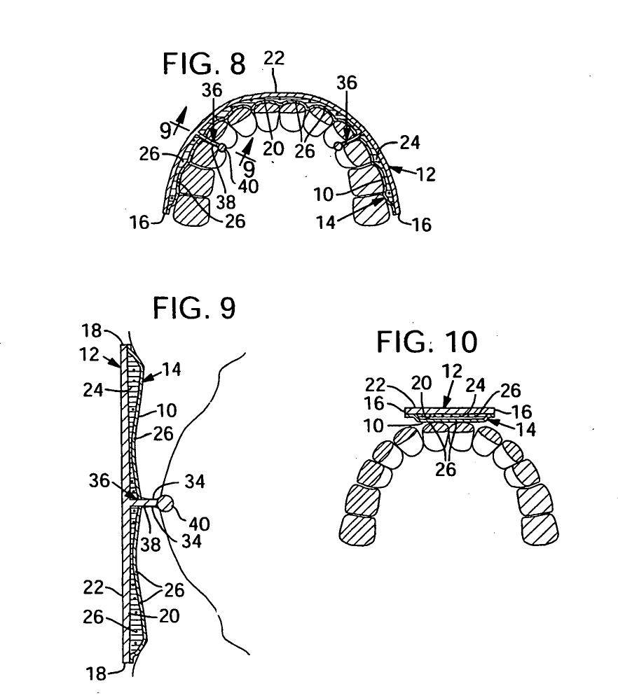

FIG. 8 shows another embodiment having retention arms 36. Retention arms 36 extend substantially perpendicularly to active surface 20 of substratum 12 in a direction toward the subject's teeth. The device can have only one retention arm 26 or can have a plurality of retention arms. Retention arms 36 can comprise a cylindrical arm portion 38 that terminates in a bulbous end 40. Cylindrical arm portion 38 can also have a cross section of any other geometric shape. Additionally, bulbous end 40 can be omitted and arm 36 may simply terminate. Arms 36 can be connected to substratum 12 by methods that are well known, for example, gluing, or heat-sealing.

FIG. 9 shows how the device might be used to treat more than one row of teeth simultaneously. Horizontal edges 18 of substratum 12 define a width wide enough to treat the upper and lower rows of a subject's teeth. A cross section of one retention arm 36 can be seen in the subject's mouth between the upper and lower rows of teeth. Permeable layer 14 can be designed with pre-configured holes sized to allow retention arms 36 to pass through the permeable layer as the permeable layer is attached to substratum 12, but also sized to ensure a snug fit around the retention arms to prevent the leakage of treatment layer 24. If the fit is not snug, a sealing agent such as glue can be used to prevent leakage by sealing permeable layer 14 to retention arms 36. The swelling of treatment layer 24 and the stretching of permeable layer 14 bring the active surface 10 of permeable layer and treatment agent 26 closer to the subject's teeth as shown.

FIG. 10 shows another embodiment where the device is used without conforming it to the subject's teeth. The horizontal cross section of the device is substantially straight. The distance between its vertical edges 16 is wide enough to cover only two teeth of a row of teeth. In other embodiments the distance between vertical edges 16 can be wide enough to cover only one tooth or wide enough to cover a plurality of teeth. Additionally, the width defined by the horizontal edges (not shown) can be wide enough to cover a single row or both rows of the subject's teeth. The device can be held in place by a part of the subject's body, for example, the lips.

In view of the many possible embodiments that the disclosed device can take, it will be recognized that the specific illustrations disclosed herein should not be interpreted to limit the scope of the following claims. Rather, the scope of the following claims is in accord with their broadest meaning in light of this disclosure.

I claim:

1. A device for the treatment of teeth comprising:
a substratum comprising a curviplaner band of non-porous material;
a permeable layer attached to the substratum; and
a treatment layer disposed between the substratum and the permeable layer comprising a treatment agent, wherein the permeable layer allows the treatment agent to pass through the permeable layer after an aqueous solution is introduced to the device, and wherein the permeable layer does not allow a substantial portion of the treatment agent to pass through the permeable layer before the aqueous solution is introduced.

2. The device of claim 1 wherein the substratum is pre-formed into an arcuate shape.

3. The device of claim 1 wherein the substratum is flat, but is conformable to an arcuate shape.

4. The device of claim 1 wherein the device is adaptable to fit a range of variously sized sets of teeth.

5. The device of claim 1 wherein the substratum has horizontal edges defining a width of the substratum and the teeth to be treated have buccal and lingual surfaces, wherein the width of the substratum is sufficient to cover at least a portion of only the buccal or lingual surface of a single row of teeth.

6. The device of claim 1 wherein the substratum has horizontal edges defining a width of the substratum and the teeth to be treated have buccal and lingual surfaces, wherein the width of the substratum is sufficient to cover at least a portion of only the buccal or lingual surfaces of both upper and lower rows of teeth.

7. The device of claim 1 wherein the substratum is a water permeable material.

8. The device of claim 3 wherein the conformable substratum is permanently deformable.

9. The device of claim 3 wherein the conformable substratum is not permanently deformable.

10. The device of claim 1 further comprising a retention member attached to the substratum.

11. The device of claim 10 wherein the substratum has an active surface, and wherein the retention member comprises at least one retention arm extending substantially perpendicularly from the active surface of the substratum.

12. The device of claim 10 wherein the substratum has an active surface, and wherein the retention member comprises an extension of the substratum in a direction faced by the active surface of the substratum.

13. The device of claim 1 wherein the permeable layer is stretchable to accommodate expansion of the treatment layer.

14. The device of claim 1 wherein the permeable layer is a perforated plastic sheet.

15. The device of claim 1 wherein the permeable layer is a fabric permeable to the treatment agent.

16. The device of claim 15 wherein the fabric comprises polypropylene fibers.

17. The device of claim 1 wherein the treatment agent is a dry powder.

18. The device of claim 17 wherein the dry powder is a redox agent.

19. The device of claim 18 wherein the redox agent is selected from the group consisting of monopersulfate, Oxone, ammonium persulfate, potassium persulfate, potassium monopersulfate, potassium peroxymonosulfate, potassium bisulfatc, potassium sulfate and potassium peroxidisulfate.

20. The device of claim 1 wherein the treatment agent is a bleaching agent of the peroxide class in dry form.

21. The device of claim 20 wherein the bleaching agent is selected from the group consisting of hydrogen peroxide, carbamide peroxide, urea peroxide, sodium percarbonate, sodium perborate, calcium hydroxide, potassium chlorate, magnesium carbonate and perhydrol urea.

22. The device of claim 1 wherein the treatment layer further comprises a gelling agent in dry form.

23. The device of claim 22 wherein the gelling agent is selected from the group consisting of an alpha starch, an agar, hydroxyethyl cellulose, mangrot seed, hydroxymethyl cellulose and fumed aluminum silicate.

24. The device of claim 1 wherein the treatment layer further comprises a dry, solid, water-insoluble, physiologically unobjectionable water-swellable agent.

25. The device of claim 24 wherein the water-swellable agent is a polymeric sorbent.

26. The device of claim 25 wherein the polymeric sorbent is selected from the group consisting of sodium polyacrylate, sodium polyacrylamide, poly-N-vinylpyrrolidone, poly-vinyltoluenesulfonate, poly-sulfoethyl acrylate, poly-2-hydroxyethyl acrylate, poly-vinylmethyloxazolidinone, hydrolyzed polyacrylamide, polyacrylic acid, copolymers of acrylamide and acrylic acid and alkali metal salts of the polymers as contain sulfonate or carboxylate groups.

27. The device of claim 24 wherein the water swellable agent is a naturally occurring water-swellable agent.

28. The device of claim 27 wherein the naturally occurring water swellable agent is selected from the group consisting of mangrot seed, ground root of the bunk plant, cotton, and sponge.

29. The device of claim 1 wherein the treatment layer further comprises a palliative agent for periodontal tissues in dry form.

30. The device of claim 29 wherein the palliative agent is selected from the group consisting of aloe, eugenol, corticosteroid and vitamin E.

31. The device of claim 1 wherein the treatment layer further comprises an agent for administering fluoride in dry form.

32. The device of claim 1 wherein the treatment layer further comprises an agent to decrease tooth sensitivity in dry form.

33. The device of claim 32 wherein the agent to decrease tooth sensitivity is derived from a member of the group consisting of potassium nitrate, citric acid, citric acid salts, sodium fluoride, and strontium chloride.

34. The device of claim 1 wherein the treatment layer further comprises a flavoring agent in dry form.

35. The device of claim 1 wherein the treatment layer further comprises an anti-microbial agent in dry form.

36. The device of claim 35 wherein the anti-microbial agent is selected from the group consisting of chlorhexadine, tetracycline, cetyl pyridinium chloride, benzalkonium chloride, cetyl pyridinium chloride, cetyl pyridinium bromide, methyl benzoate, and proply benzoate.

37. A device for the treatment of teeth comprising:
a substratum comprising a curviplaner band of non-porous material;
a retaining layer of material attached to the substratum;
a permeable layer of material attached to the retaining layer; and
a treatment layer disposed between the retaining layer and the permeable layer, wherein the treatment layer comprises a dry, solid powder bleaching agent.

38. The device of claim 37 wherein the substratum has an active surface, and wherein the retaining layer has an active and a non-active surface, further comprising a layer of adhesive adhered to the non-active surface of the retaining layer, wherein the retaining layer is removably attached to the active surface of the substratum by the layer of adhesive.

39. A device for the treatment of teeth comprising:
A treatment agent;
treatment agent delivery means for delivering a treatment agent to a subject's teeth; and
curviplaner support means for positioning the treatment agent delivery means against or in proximity to teeth;
wherein the treatment agent delivery means is attached to the support means to envelop the treatment agent.

40. A method for bleaching the lingual or buccal surfaces of the teeth of a subject comprising:
providing a device including a non-porous substratum, a permeable layer attached to the active surface of the substratum, and a dry, solid powder bleaching agent disposed between the substratum and the permeable layer; adding an aqueous solution to the device; and placing the device against only the lingual or the buccal surfaces of the teeth of the subject before or after the aqueous solution is added.

41. The method of claim 40 wherein the device is placed against the teeth of the subject before addition of the aqueous solution, and wherein the aqueous solution comprises saliva.

42. A device for the treatment of teeth comprising:
a substratum comprising a curviplaner band of non-porous polymeric material;
a permeable layer attached to the active surface of the substratum; and
a treatment layer disposed between the substratum and the permeable layer comprising a treatment agent, wherein the treatment agent is a composition of a bleaching agent and a water swellable agent in dry form that is free between the substratum and permeable layer, wherein the permeable layer allows the treatment agent to pass through the permeable layer after an aqueous solution is introduced to the device, and wherein the permeable layer does not allow a substantial portion of the treatment agent to pass through the permeable layer before the aqueous solution is introduced.
